(19) United States
(12) Patent Application Publication
(10) Pub. No.: US 2022/0104708 A1
(43) Pub. Date: Apr. 7, 2022

(54) MAGNIFYING GLASS FOR ACQUIRING SKIN IMAGE COMPENSATED FOR DISTORTION

(71) Applicant: F&D PARTNERS INC., Sejong-si (KR)

(72) Inventor: Hyunjae ZHANG, Daejeon (KR)

(73) Assignee: F&D PARTNERS INC., Sejong-si (KR)

(21) Appl. No.: 17/109,206

(22) Filed: Dec. 2, 2020

(30) Foreign Application Priority Data
Oct. 7, 2020 (KR) 10-2020-0129096

(51) Int. Cl. A61B 5/00; G02B 25/02; G02B 25/00

(52) U.S. Cl. CPC A61B 5/0077; G02B 25/02; G02B 5/3025; A61B 5/441; G02B 25/007

(57) ABSTRACT

Disclosed is a magnifying glass for skin diagnosis capable of acquiring a distortion-compensated skin image, and more particularly a magnifying glass for skin diagnosis capable of acquiring a magnified skin image and at the same time compensating for distortion occurring at the time of acquisition of the magnified skin image to provide an observer with a distortion-compensated, magnified skin image, thereby improving visibility, and therefore the observer can more clearly and vividly observe the skin. The magnifying glass includes an optical/light radiation structure, a light emission controller, and a housing.

### BACKGROUND OF THE INVENTION

### Field of the Invention

The present invention relates to a magnifying glass for skin diagnosis capable of acquiring a distortion-compensated skin image, and more particularly to a magnifying glass for skin diagnosis capable of acquiring a magnified skin image and at the same time compensating for distortion occurring at the time of acquisition of the magnified skin image to provide an observer with a distortion-compensated, magnified skin image, thereby improving visibility, and therefore the observer can more clearly and vividly observe the skin.

### Description of the Related Art

A dermatoscope device is a diagnostic tool used to observe pigmented lesions in the epidermis of the skin and the dermis of the mammilla, which are difficult to observe with the naked eye, in order to discriminate lesions such as malignant melanoma. In addition to such pigmented skin lesions, the dermatoscope device is also used to diagnose epidermis tumors, papulosquamous diseases, and nail lesions and to inspect parasitic insects on the skin.

In other words, the dermatoscope device provides much more than information, based on which diagnosis is made, for an observer, such as a doctor, to acquire with the naked eye before biopsy, whereby accurate diagnosis and rapid treatment are possible.

There is a need to develop technology capable of improving the resolution of the dermatoscope device and reducing distortion of the dermatoscope device to improve visibility, whereby the observer can more clearly and vividly observe a magnified image at the time of observing the surface of the skin such that the observer can accurately acquire much more information.

In addition, dermatoscope devices having various structures in consideration of portability and convenience in use thereof have been developed. In connection therewith, there is a necessity for a new type dermatoscope device that an observer can use more conveniently.

In addition, dermatoscope devices having various structures in consideration of portability and convenience in use thereof have been developed. In connection therewith, a dermatoscope device that an observer can use more conveniently and that is configured to operate together with another device is disclosed in US Patent Application Publication No. 2014-0243685 entitled DERMATOSCOPE DEVICES (hereinafter referred to as a “prior art document”).

In conventional dermatoscope devices including the above prior art document, however, optical visibility in which an observer can observe a magnified observation target is not sufficiently improved. Furthermore, in order to more clearly observe lesions on skin, whether to radiate light is decided when the light is radiated to an observation zone, or whether to polarize light is decided.

In other words, the conventional dermatoscope devices including the above prior art document have problems in that it is not possible for an observer, such as a doctor, to elaborately control light radiation conditions that are most suitable for skin conditions of patients in consideration thereof.

Therefore, there is a need for a magnifying glass for skin diagnosis capable of acquiring a magnified skin image and at the same time compensating for distortion occurring at the time of acquisition of the magnified skin image to provide an observer with a distortion-compensated, magnified skin image, thereby improving visibility, and therefore the observer can more clearly and vividly observe the skin.

### PRIOR ART DOCUMENT

### Patent Document

(Patent Document 1) US Patent Application Publication No. 2014-0243685

### SUMMARY OF THE INVENTION

The present invention has been made in view of the above problems, and it is an object of the present invention to provide a magnifying glass for skin diagnosis including a first polarizer and a second polarizer, by which cross-polarization is provided, whereby it is possible to remove diffuse reflection occurring on the surface of an observation target.

It is another object of the present invention to provide a magnifying glass for skin diagnosis including an optical lens part including a convex-lens-type optical lens and a concave-lens-type optical lens having opposite concave surfaces or having a concave surface located at an observer side, whereby it is possible to reduce distortion occurring at the time of acquiring a magnified skin image, and therefore it is possible to improve visibility, whereby the skin is more clearly and vividly observed.

In accordance with the present invention, the above and other objects can be accomplished by the provision of a magnifying glass capable of compensating for distortion that is configured to be attached to a mobile photographing device, the magnifying glass including:

an optical/light radiation structure including an optical unit configured to allow an observer to check an observation target while magnifying the observation target and a light radiation unit to which the optical unit is coupled, the light radiation unit being configured to radiate light to the observation target to be checked while being magnified through the optical unit;

a light emission controller configured to control light emission of the light radiation unit; and

a housing in which the optical/light radiation structure and the light emission controller are mounted.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a magnifying glass for skin diagnosis according to the present invention;

FIG. 2 is a sectional view of the magnifying glass for skin diagnosis according to the present invention;

FIG. 3 is a perspective view of the magnifying glass for skin diagnosis according to the present invention when viewed at another angle;

FIGS. 4 and 5 are perspective views of the magnifying glass for skin diagnosis according to the present invention when viewed at a further angle;

FIG. 6 is an interior perspective view of the magnifying glass for skin diagnosis according to the present invention;

FIG. 7 is a perspective view of a focal distance adjustment member of the magnifying glass for skin diagnosis according to the present invention;

FIG. 8 is a side view showing coupling between a light radiation unit housing and a cylindrical part of the magnifying glass for skin diagnosis according to the present invention;

FIG. 9 is a plan view showing a light radiation unit of the magnifying glass for skin diagnosis according to the present invention;

FIG. 10 is a perspective view showing coupling between the light radiation unit housing and an optical unit housing of the magnifying glass for skin diagnosis according to the present invention;

FIG. 11 is a perspective view showing the light radiation unit housing and a second polarizer of the magnifying glass for skin diagnosis according to the present invention;

FIG. 12 is an illustrative view showing the arrangement of a first polarizer and an optical lens part of the magnifying glass for skin diagnosis according to the present invention;

FIGS. 13 and 14 distortion graphs of the magnifying glass for skin diagnosis according to the present invention;

FIG. 15 is an illustrative view of a distorted image acquired by a conventional magnifying glass; and

FIG. 16 is an illustrative view of a distortion-compensated image acquired by the magnifying glass for skin diagnosis according to the present invention.

### DETAILED DESCRIPTION OF THE INVENTION

The following description merely illustrates the principle of the present invention. Therefore, those skilled in the art will invent various devices that realize the principle of the present invention and are included in the concept and scope of the present invention, although not definitely described in this specification and not shown in the accompanying drawings.

In addition, it should be understood that all conditional terms and embodiments mentioned in this specification are provided in principle only for understanding of the concept of the present invention and that the present invention is not limited to such embodiments.

A magnifying glass capable of compensating for distortion that is configured to be attached to a mobile photographing device according to an embodiment of the present invention includes:

an optical/light radiation structure including an optical unit configured to allow an observer to check an observation target while magnifying the observation target and a light radiation unit to which the optical unit is coupled, the light radiation unit being configured to radiate light to the observation target to be checked while being magnified through the optical unit;

a light emission controller configured to control light emission of the light radiation unit; and

a housing in which the optical/light radiation structure and the light emission controller are mounted.

The optical unit includes:

a first polarizer located at an observer side, the first polarizer constituting a polarization axis set in parallel in a first direction;

an optical lens part located at an observation target side of the first polarizer, the optical lens part being configured to allow the observer to check the observation target while magnifying the observation target; and

an optical unit housing having a cylindrical part formed in the center thereof,

the first polarizer and the optical lens part being provided inside the cylindrical part.

The optical lens part includes at least one of:

a first optical lens located at the observation target side of the first polarizer, the first optical lens being configured as a plano-convex lens having a convex surface located at the observation target side;

a second optical lens located at the observation target side of the first optical lens, the second optical lens being configured as a biconvex lens having opposite convex surfaces; and

a third optical lens located at the observation target side of the second optical lens, the third optical lens being configured as a biconcave lens having opposite concave surfaces or a plano-concave lens having a concave surface located at the observer side.

The radius of curvature of the convex surface of the first optical lens located at the observation target side is equal to or less than the radius of curvature of the convex surface of the second optical lens located at the observer side, and is equal to or greater than the radius of curvature of the convex surface of the second optical lens located at the observation target side.

In the case in which the third optical lens is configured as a biconcave lens, the radius of curvature of the convex surface of the second optical lens located at the observation target side is equal to the radius of curvature of the concave surface of the third optical lens located at the observer side and the radius of curvature of the concave surface of the third optical lens located at the observation target side.

In the case in which the third optical lens is configured as a plano-concave lens having a concave surface located at the observer side, the radius of curvature of the convex surface of the second optical lens located at the observation target side is equal to the radius of curvature of the concave surface of the third optical lens located at the observer side.

The radius of curvature of the convex surface of the second optical lens located at the observation target side is equal to or less than the radius of curvature of the convex surface of the second optical lens located at the observer side.

In the case in which the third optical lens is configured as a biconcave lens, the radius of curvature of the concave surface of the third optical lens located at the observation target side is equal to or less than the radius of curvature of the convex surface of the second optical lens located at the observation target side.

The third optical lens is provided in order to reduce distortion of the photographing device.

The light radiation unit includes:

a doughnut-shaped light emission board including a plurality of first light emission parts formed on an outer layer so as to be spaced apart from each other by a predetermined distance, the plurality of first light emission parts being configured to simultaneously emit light in response to a first light emission signal from the light emission controller and a plurality of second light emission parts formed on an inner layer formed inside the outer layer so as to be spaced apart from each other by a predetermined distance, the plurality of second light emission parts being configured to simultaneously emit light in response to a second light emission signal from the light emission controller;

a second polarizer located in a direction in which light emitted by the first light emission parts located on the outer layer is radiated or in a direction in which light emitted by the second light emission parts located on the inner layer is radiated, the second polarizer constituting a polarization axis set in a second direction perpendicular to the first direction defined by the first polarizer; and

a light radiation unit housing in which the light emission board and the second polarizer are mounted.

The light radiation unit housing includes:

a plurality of coupling projecting parts formed at a side of the light radiation unit housing so as to be spaced apart from each other by a predetermined distance such that an end of the optical unit housing is detachably coupled to the plurality of coupling projecting parts;

a light emission hole formation part having a plurality of light emission holes formed inside the light radiation unit housing at positions corresponding to the plurality of first light emission parts and the plurality of second light emission parts of the light emission board, the plurality of light emission holes being formed so as to be spaced apart from each other by a predetermined distance; and

a plurality of seating projecting parts formed at the observer side of the light emission hole formation part so as to be spaced apart from each other by a predetermined distance, the plurality of seating projecting parts being configured to seat the second polarizer.

Cross-polarization is provided by the first polarizer and the second polarizer in order to remove diffuse reflection occurring on the surface of the observation target.

A button is formed at the housing. Upon receiving a manipulation signal input through the button, the light emission controller provides a first light emission signal or a second light emission signal to the plurality of first light emission parts or to the plurality of second light emission parts in order to operate the plurality of first light emission parts or the plurality of second light emission parts.

Alternatively, upon receiving a manipulation signal from a smart device through wireless communication with the smart device, the light emission controller provides a first light emission signal or a second light emission signal to the plurality of first light emission parts or to the plurality of second light emission parts in response to the manipulation signal in order to operate the plurality of first light emission parts or the plurality of second light emission parts. The smart device may be a smartphone.

Hereinafter, an embodiment of the magnifying glass capable of compensating for distortion that is configured to be attached to the mobile photographing device according to the present invention will be described in detail with reference to the accompanying drawings.

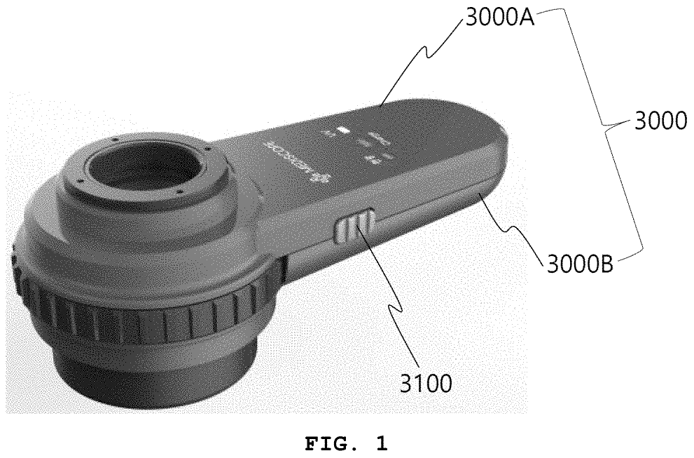

As shown in FIG. 1, the magnifying glass capable of compensating for distortion that is configured to be attached to the mobile photographing device according to the present invention includes an optical/light radiation structure 1000, a light emission controller 2000, and a housing 3000.

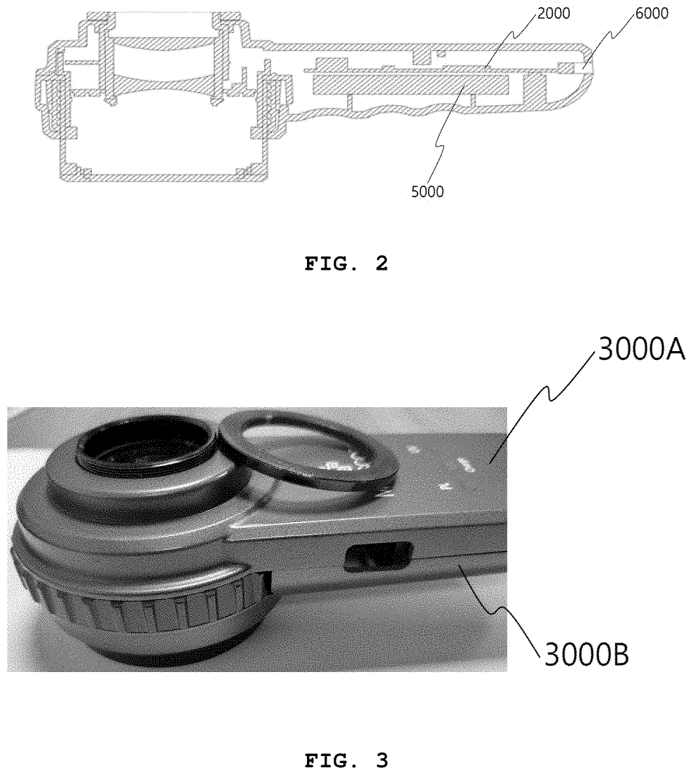

The housing 3000 is a main body in which the optical/light radiation structure 1000, the light emission controller 2000, and other components, such as a button 3100, a focal distance adjustment member 4000, a battery 5000, and a charging port 6000, are mounted. As shown in FIGS. 1 to 4, a space is defined in the housing 3000 as the result of coupling between a first housing part 3000A located at an observer side and a second housing part 3000B located at an observation target side.

Here, the housing 3000 is formed in the shape of a handle such that an observer can hold the housing in order to use the magnifying glass. The light emission controller 2000 and the battery 5000 are mounted in the housing 3000, and the button 3100, the charging port 6000, the optical/light radiation structure 1000, and the focal distance adjustment member 4000 are coupled to one side of the housing 3000 such that the observer observes the surface of an observation target, i.e. skin.

The button 3100 is formed at the housing 3000. Upon receiving a manipulation signal input through the button 3100, the light emission controller 2000 provides a first light emission signal or a second light emission signal to the plurality of first light emission parts 211 or to the plurality of second light emission parts 212 in order to operate the plurality of first light emission parts 211 or the plurality of second light emission parts 212.

Specifically, as shown in FIG. 1, the button 3100 is pushed or touched by the observer to generate a manipulation signal and provides the manipulation signal to the light emission controller 2000.

In the embodiment of the present invention, buttons may be formed at the left side and the right side of the housing. When the left button is pushed, a first light emission signal may be provided to the plurality of first light emission parts. When the right button is pushed, a second light emission signal may be provided to the plurality of second light emission parts.

The light emission controller 2000 controls light emission of the light radiation unit 200.

Specifically, upon receiving a manipulation signal input through the button 3100 formed at the housing 3000, the light emission controller 2000 provides a first light emission signal or a second light emission signal to the plurality of first light emission parts 211 or to the plurality of second light emission parts 212 in response to the manipulation signal in order to operate the plurality of first light emission parts 211 or the plurality of second light emission parts 212.

In another embodiment, upon receiving a manipulation signal from a smart device through wireless communication with the smart device, the light emission controller provides a first light emission signal or a second light emission signal to the plurality of first light emission parts 211 or to the plurality of second light emission parts 212 in response to the manipulation signal in order to operate the plurality of first light emission parts 211 or the plurality of second light emission parts 212. The smart device may be a smartphone.

Hereinafter, the optical/light radiation structure 1000 will be described.

The optical/light radiation structure 1000 includes:

an optical unit 100 configured to allow the observer to check the observation target while magnifying the observation target; and

a light radiation unit 200 to which the optical unit 100 is coupled, the light radiation unit 200 being configured to radiate light to the observation target to be checked while being magnified through the optical unit 100.

That is, the optical/light radiation structure 1000 is a substantial component that realizes optical properties and functions of the magnifying glass for skin diagnosis according to the present invention through the optical unit 100 configured to allow the observer to check the observation target while magnifying the observation target and the light radiation unit 200 configured to radiate light to the observation target to be checked while being magnified through the optical unit 100.

Light radiation (light emission) through the light radiation unit 200 of the optical/light radiation structure 1000 is controlled by the light emission controller 2000.

Power necessary to operate the light radiation unit 200 and the light emission controller 2000 is provided by the battery 5000 mounted in the housing 3000. The charging port 6000 is formed at the housing 3000 in order to charge the battery 5000 with electricity.

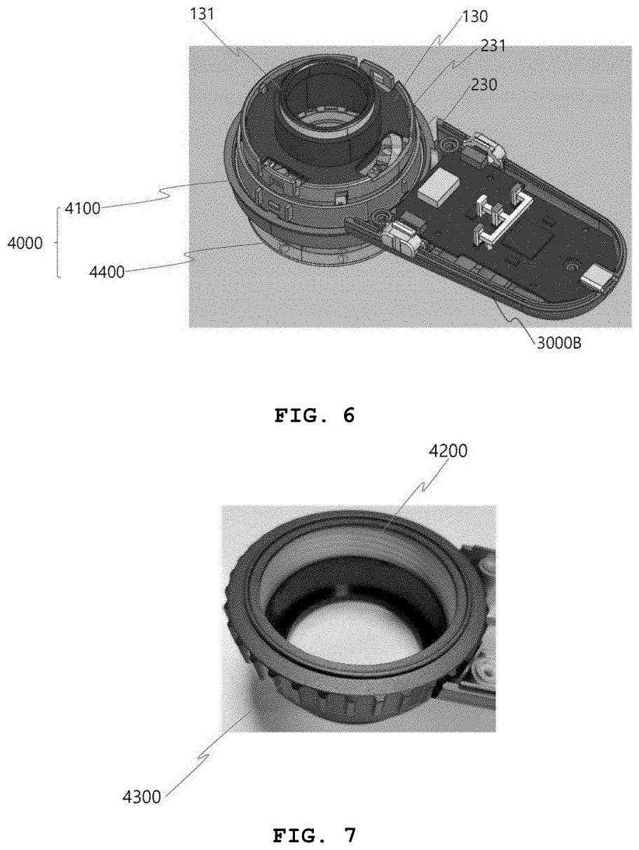

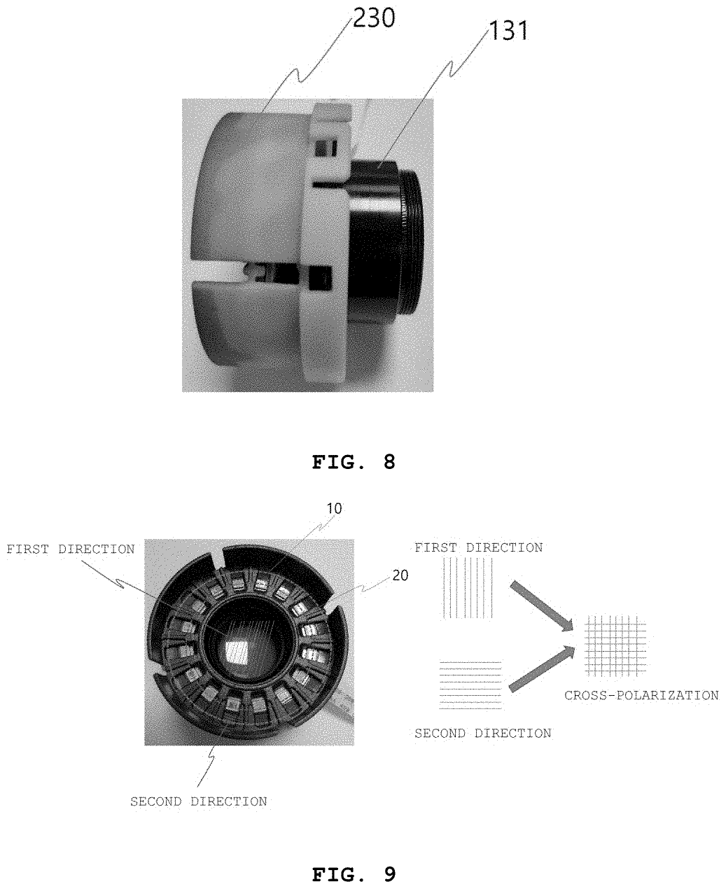

Specifically, as shown in FIGS. 6 and 10, the optical unit 100 includes:

a first polarizer 110 located at the observer side, the first polarizer constituting a polarization axis set in parallel in a first direction;

an optical lens part 120 located at the observation target side of the first polarizer, the optical lens part being configured to allow the observer to check the observation target while magnifying the observation target; and

an optical unit housing 130 having a cylindrical part 131 formed in the center thereof.

That is, as shown in FIGS. 6 and 10, the optical unit 100 includes an optical unit housing 130 having a cylindrical part 131 formed in the center thereof, and the first polarizer 110 and the optical lens part 120 are provided inside the cylindrical part 131.

The first polarizer 110 is located at the observer side and constitutes a polarization axis set in parallel in the first direction.

The optical lens part 120 is located at the observation target side of the first polarizer and allows the observer to check the observation target while magnifying the observation target.

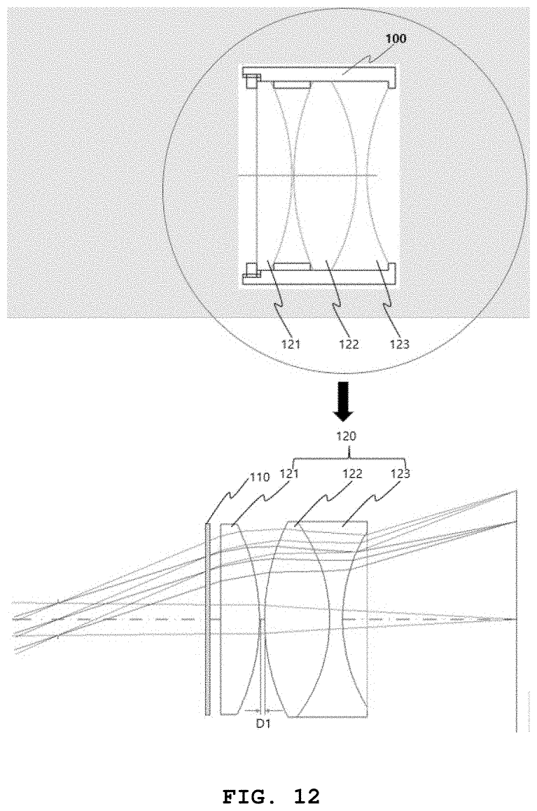

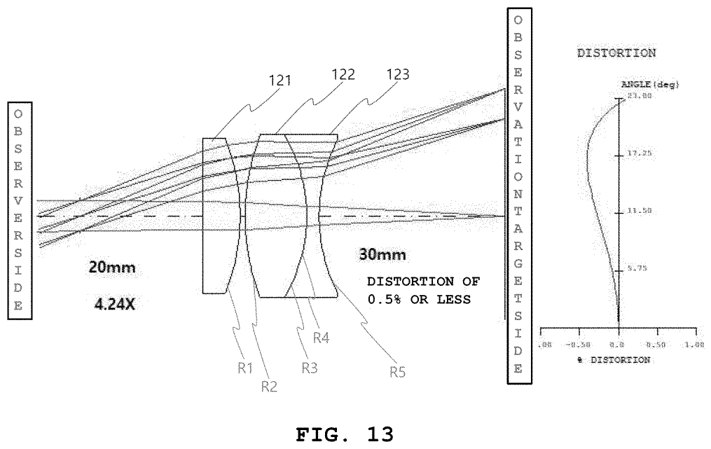

At this time, as shown in FIG. 12, the optical lens part 120 includes at least one of:

a first optical lens 121 located at the observation target side of the first polarizer, the first optical lens 121 being configured as a plano-convex lens having a convex surface located at the observation target side;

a second optical lens 122 located at the observation target side of the first optical lens, the second optical lens 122 being configured as a biconvex lens having opposite convex surfaces; and

a third optical lens 123 located at the observation target side of the second optical lens, the third optical lens 123 being configured as a biconcave lens having opposite concave surfaces or a plano-concave lens having a concave surface located at the observer side.

Specifically, the first optical lens 121 is located at the observation target side of the first polarizer, and is configured as a plano-convex lens having a convex surface located at the observation target side.

The second optical lens 122 is located at the observation target side of the first optical lens. The first optical lens 121 and the second optical lens 122 are preferably disposed such that the central axis of the first optical lens 121 and the central axis of the second optical lens 122 are spaced apart from each other by a predetermined distance Dl.

At this time, the second optical lens 122 is preferably configured as a biconvex lens having opposite convex surfaces.

The third optical lens 123 is located at the observation target side of the second optical lens, and is configured as a biconcave lens having opposite concave surfaces or a plano-concave lens having a concave surface located at the observer side.

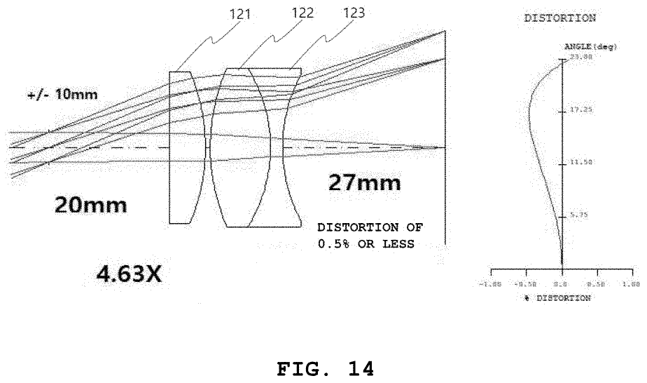

Specifically, as shown in FIGS. 13 and 14, the radius of curvature R1 of the convex surface of the first optical lens 121 located at the observation target side is equal to or less than the radius of curvature R2 of the convex surface of the second optical lens 122 located at the observer side, and is equal to or greater than the radius of curvature R3 of the convex surface of the second optical lens 122 located at the observation target side.

For example, on the assumption that R1 is about 30 mm, R2 may be about 35 mm, and R3 may be about 25 mm.

In the case in which the third optical lens 123 is configured as a biconcave lens, the radius of curvature R3 of the convex surface of the second optical lens 122 located at the observation target side is equal to the radius of curvature R4 of the concave surface of the third optical lens 123 located at the observer side and the radius of curvature R5 of the concave surface of the third optical lens 123 located at the observation target side.

For example, on the assumption that the radius of curvature R3 of the convex surface of the second optical lens 122 located at the observation target side is about 25 mm, both the radius of curvature R4 of the concave surface of the biconcave-lens-type third optical lens 123 located at the observer side and the radius of curvature R5 of the concave surface of the biconcave-lens-type third optical lens 123 located at the observation target side may be about 25 mm.

Alternatively, in the case in which the third optical lens 123 is configured as a plano-concave lens having a concave surface located at the observer side, the radius of curvature R3 of the convex surface of the second optical lens 122 located at the observation target side is equal to the radius of curvature R4 of the concave surface of the third optical lens 123 located at the observer side.

For example, on the assumption that the radius of curvature R3 of the convex surface of the second optical lens 122 located at the observation target side is about 25 mm, the radius of curvature R4 of the concave surface of the plano-concave-lens-type third optical lens 123 located at the observer side may be about 25 mm.

The reason that the radii of curvature are set and the lenses are arranged as described above is that it is necessary to compensate for distortion due to a wide-angle lens.

For example, in the case of a magnifying power of 4.24, as shown in FIG. 13, the distance from the observer side to the lens is 20 mm, the distance from the lens to the observation target side is 30 mm, and distortion is 0.5% or less, whereby it is possible to provide a clear image.

Also, in the case of a magnifying power of 4.63, as shown in FIG. 14, the distance from the observer side to the lens is 20 mm, the distance from the lens to the observation target side is 27 mm, and distortion is 0.5% or less, whereby it is possible to provide a clear image.

At this time, the present invention is technically characterized in that the third optical lens 123 is configured as a concave lens.

The reason for this is that, when the magnifying glass is coupled to the photographing device, e.g. a smartphone, and the surface of the observation target, i.e. skin, is checked through the smartphone, distortion occurs due to a wide-angle lens, whereby it is not possible to provide a clear image.

Therefore, the third optical lens 123 is configured as a concave lens in order to reduce distortion occurring due to a wide-angle lens, i.e. in order to compensate for distortion.

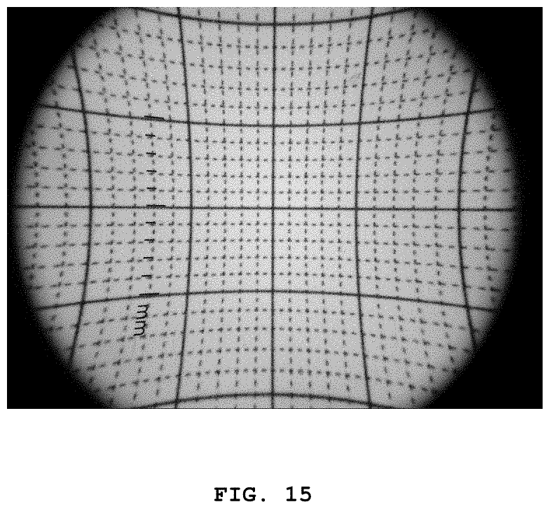

Experiments were carried out in order to support the above effect. A photographing device having a wide-angle lens was mounted to a general magnifying glass, and a two-dimensional compensation sample was magnified and observed. As a result, it can be seen that considerable distortion occurred, as shown in FIG. 15.

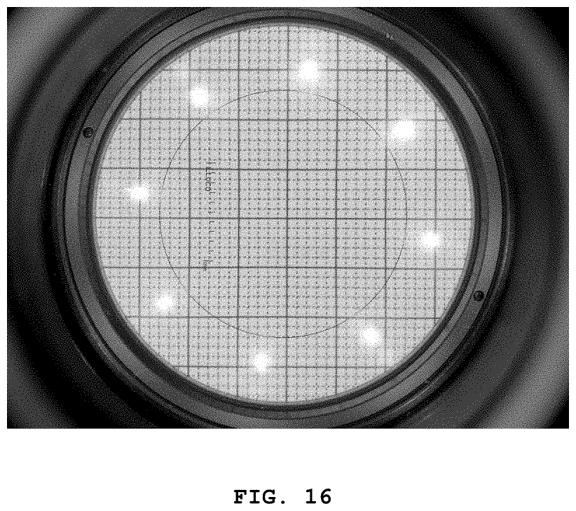

A photographing device having a wide-angle lens was mounted to the magnifying glass according to the present invention, and a two-dimensional compensation sample was magnified and observed. As a result, it can be seen that distortion hardly occurred, as shown in FIG. 16. Therefore, the experiments reveal that the present invention has remarkable effects.

Meanwhile, in another embodiment, the radius of curvature R3 of the convex surface of the second optical lens 122 located at the observation target side is equal to or less than the radius of curvature R2 of the convex surface of the second optical lens 122 located at the observer side.

Meanwhile, in a further embodiment, in the case in which the third optical lens 123 is configured as a biconcave lens, the radius of curvature R5 of the concave surface of the third optical lens 123 located at the observation target side is equal to or less than the radius of curvature R3 of the convex surface of the second optical lens 122 located at the observation target side.

The above configuration is provided to exhibit a synergistic effect of compensating for a distortion phenomenon in a super wide angle having a wider visible range than a general wide angle.

As shown in FIG. 11, the light radiation unit 200 includes a doughnut-shaped light emission board 210, a second polarizer 220, and a light radiation unit housing 230.

Specifically, the light radiation unit 200 includes:

a doughnut-shaped light emission board 210 including a plurality of first light emission parts 211 formed on an outer layer so as to be spaced apart from each other by a predetermined distance, the plurality of first light emission parts 211 being configured to simultaneously emit light in response to a first light emission signal from the light emission controller 2000 and a plurality of second light emission parts 212 formed on an inner layer formed inside the outer layer so as to be spaced apart from each other by a predetermined distance, the plurality of second light emission parts 212 being configured to simultaneously emit light in response to a second light emission signal from the light emission controller 2000;

a second polarizer 220 located in a direction in which light emitted by the first light emission parts located on the outer layer is radiated or in a direction in which light emitted by the second light emission parts located on the inner layer is radiated, the second polarizer 220 constituting a polarization axis set in a second direction perpendicular to the first direction defined by the first polarizer 110; and

a light radiation unit housing 230 in which the light emission board 210 and the second polarizer 220 are mounted.

The light emission board 210 is configured to have a doughnut shape, and includes a plurality of first light emission parts 211 and a plurality of second light emission parts 212.

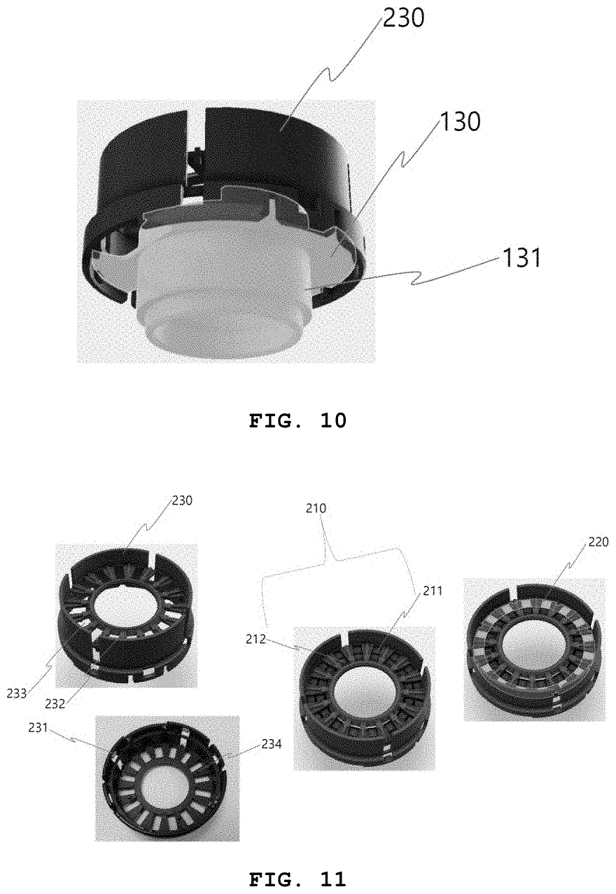

Specifically, as shown in FIG. 9, the plurality of first light emission parts 211 is formed on the outer layer 10 so as to be spaced apart from each other by a predetermined distance, and simultaneously emits light in response to a first light emission signal from the light emission controller 2000.

The plurality of second light emission parts 212 is formed on the inner layer 20 formed inside the outer layer so as to be spaced apart from each other by a predetermined distance, and simultaneously emits light in response to a second light emission signal from the light emission controller 2000.

A light source configured to provide various wavelength bands, such as LED, UV, and IR, may be adopted as each of the first light emission parts or each of the second light emission parts, and the wavelength band of the light source is not limited to a specific wavelength band.

Consequently, it is possible to acquire images of the surfaces of various observation targets, i.e. various skins, whereby it is possible to accurately examine the state of the surface of each skin.

Specifically, as shown in FIG. 11, the second polarizer 220 is located in a direction in which light emitted by the first light emission parts located on the outer layer is radiated, or is located in a direction in which light emitted by the second light emission parts located on the inner layer is radiated.

FIG. 11 shows an example in which the second polarizer is located in a direction in which light emitted by the first light emission parts located on the outer layer is radiated.

Consequently, in the case in which the second polarizer is located at the above position in a doughnut shape, as described above, the second polarizer 220 constitutes a polarization axis set in a second direction perpendicular to the first direction defined by the first polarizer 110.

As a result, cross-polarization is provided by the first polarizer 110 and the second polarizer 120, whereby it is possible in order to remove diffuse reflection occurring on the surface of the observation target.

Specifically, the magnifying glass provides illumination to the surface of the skin to be observed, and therefore diffuse reflection inevitably occurs due to the illumination.

In order to remove this, therefore, a polarization filter is used, and cross-polarization is provided through the above structure.

The doughnut-shaped light emission board 210 and the second polarizer 220 are mounted in the light radiation unit housing 230.

Specifically, as shown in FIGS. 6, 8, 10, and 11, the light radiation unit housing 230 includes:

a plurality of coupling projecting parts 231 formed at a side of the light radiation unit housing 230 so as to be spaced apart from each other by a predetermined distance such that an end of the optical unit housing 130 is detachably coupled to the plurality of coupling projecting parts;

a light emission hole formation part 232 having a plurality of light emission holes 233 formed inside the light radiation unit housing 230 at positions corresponding to the plurality of first light emission parts 211 and the plurality of second light emission parts 212 of the light emission board 210, the plurality of light emission holes 233 being formed so as to be spaced apart from each other by a predetermined distance; and

a plurality of seating projecting parts 234 formed at the observer side of the light emission hole formation part 232 so as to be spaced apart from each other by a predetermined distance, the plurality of seating projecting parts 234 being configured to seat the second polarizer 220.

That is, a plurality of coupling projecting parts 231 is formed at an end of the inside of the light radiation unit housing 230, and the optical unit housing 130 is pushed so as to be inserted into inside the coupling projecting parts so as to be coupled thereto.

In addition, a light emission hole formation part 232 is formed so as to have a plurality of light emission holes 233 formed inside the light radiation unit housing 230 at positions corresponding to the plurality of first light emission parts 211 and the plurality of second light emission parts 212 of the light emission board 210, the plurality of light emission holes 233 being formed so as to be spaced apart from each other by a predetermined distance.

At this time, a central hole is formed in the center of the light emission hole formation part 232.

In addition, a plurality of seating projecting parts 234 is formed at the observer side of the light emission hole formation part so as to be spaced apart from each other by a predetermined distance in order to seat the doughnut-shaped second polarizer.

Meanwhile, the magnifying glass capable of compensating for distortion that is configured to be attached to the mobile photographing device according to the present invention may further include a focal distance adjustment member 4000.

The focal distance adjustment member 4000 zooms in or zooms out on the observation target to be observed by the observer through the optical unit.

Specifically, the focal distance adjustment member 4000 includes:

a wheel coupling body 4100 coupled to the second housing part 3000B located at the observation target side;

a screw groove 4200 formed in the inner surface of the wheel coupling body such that a barrel 4400 is screw-engaged with the screw groove;

a focal distance adjustment wheel 4300 coupled to the outer surface of the wheel coupling body, the focal distance adjustment wheel being configured to move the barrel 4400 forwards or rearwards so as to be close to or distant from the observation target in order to perform zooming in or zooming out on the observation target; and

the barrel 4400 being screw-engaged with the screw groove, the barrel being configured to move forwards or rearwards along the screw groove in response to zooming in or zooming out of the focal distance adjustment wheel.

When the focal distance adjustment wheel 4300 is rotated, therefore, the barrel 4400 moves forwards or rearwards along the screw groove so as to be close to or distant from the observation target, whereby zooming in or zooming out is performed on the observation target.

The focal distance adjustment member 4000 according to the present invention corresponds to an embodiment, and is generally a basic component provided in the magnifying glass. Consequently, it is obvious that the operation of the focal distance adjustment member will be understood only through the above description thereof.

Meanwhile, a plurality of magnets 4410 and a cover glass 4420 are provided at the barrel 4400.

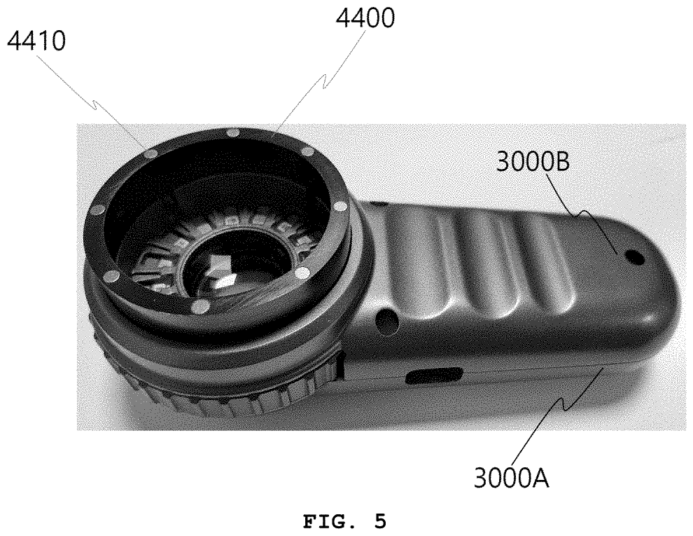

As shown in FIG. 5, the plurality of magnets 4410 is formed around an end surface of the barrel 4400, and the cover glass 4420 is attached to the magnets 4410 by magnetic force thereof.

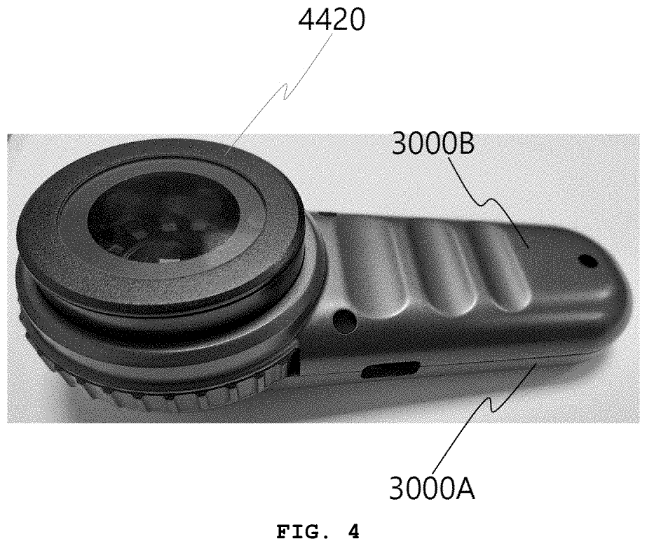

As shown in FIG. 4, the cover glass 4420 is configured to be detachably attached to the magnets 4410.

The reason that the cover glass 4420 is configured to be detachably attached to the magnets 4410 is that it is possible to softly push the surface of the observation target, i.e. skin, at the time of skin observation and to easily disinfect the cover glass after skin observation.

After skin observation, the cover glass located in contact with the surface of the observation target, i.e. skin, must be disinfected for next observation. For easy disinfection, the cover glass 4420 is detachably attached to the magnets by magnetic force thereof.

As is apparent from the above description, a magnifying glass for skin diagnosis according to the present invention includes a first polarizer and a second polarizer, by which cross-polarization is provided, whereby it is possible to remove diffuse reflection occurring on the surface of an observation target.

In addition, the magnifying glass for skin diagnosis according to the present invention includes an optical lens part including a convex-lens-type optical lens and a concave-lens-type optical lens having opposite concave surfaces or having a concave surface located at an observer side, whereby it is possible to reduce distortion occurring at the time of acquiring a magnified skin image, and therefore it is possible to improve visibility, whereby the skin is more clearly and vividly observed.

It will be apparent that, although the preferred embodiments have been shown and described above, the present invention is not limited to the above-described specific embodiments, and various modifications and variations can be made by those skilled in the art without departing from the gist of the appended claims. Thus, it is intended that the modifications and variations should not be understood independently of the technical spirit or prospect of the present invention.

What is claimed is:

1. A magnifying glass for skin diagnosis capable of acquiring a distortion-compensated skin image, the magnifying glass comprising:
an optical/light radiation structure comprising an optical unit configured to allow an observer to check an observation target while magnifying the observation target and a light radiation unit to which the optical unit is coupled, the light radiation unit being configured to radiate light to the observation target to be checked while being magnified through the optical unit;
a light emission controller configured to control light emission of the light radiation unit; and
a housing in which the optical/light radiation structure and the light emission controller are mounted.

2. The magnifying glass according to claim 1, wherein the optical unit comprises:
a first polarizer located at an observer side, the first polarizer constituting a polarization axis set in parallel in a first direction;
an optical lens part located at an observation target side of the first polarizer, the optical lens part being configured to allow the observer to check the observation target while magnifying the observation target; and
an optical unit housing having a cylindrical part formed in the center thereof,
the first polarizer and the optical lens part being provided inside the cylindrical part.

3. The magnifying glass according to claim 2, wherein the optical lens part comprises at least one of:
a first optical lens located at the observation target side of the first polarizer, the first optical lens being configured as a plano-convex lens having a convex surface located at the observation target side;
a second optical lens located at the observation target side of the first optical lens, the second optical lens being configured as a biconvex lens having opposite convex surfaces; and
a third optical lens located at the observation target side of the second optical lens, the third optical lens being configured as a biconcave lens having opposite concave surfaces or a plano-concave lens having a concave surface located at the observer side.

4. The magnifying glass according to claim 3, wherein
a radius of curvature of the convex surface of the first optical lens located at the observation target side is equal to or less than a radius of curvature of the convex surface of the second optical lens located at the observer side, and is equal to or greater than a radius of curvature of the convex surface of the second optical lens located at the observation target side,
in a case in which the third optical lens is configured as a biconcave lens, the radius of curvature of the convex surface of the second optical lens located at the observation target side is equal to a radius of curvature of the concave surface of the third optical lens located at the observer side and a radius of curvature of the concave surface of the third optical lens located at the observation target side, and
in a case in which the third optical lens is configured as a plano-concave lens having a concave surface located at the observer side, the radius of curvature of the convex surface of the second optical lens located at the observation target side is equal to the radius of curvature of the concave surface of the third optical lens located at the observer side.

5. The magnifying glass according to claim 3, wherein
a radius of curvature of the convex surface of the second optical lens located at the observation target side is equal to or less than a radius of curvature of the convex surface of the second optical lens located at the observer side, and
in a case in which the third optical lens is configured as a biconcave lens, a radius of curvature of the concave surface of the third optical lens located at the observation target side is equal to or less than the radius of curvature of the convex surface of the second optical lens located at the observation target side.

6. The magnifying glass according to claim 3, wherein the third optical lens is provided in order to reduce distortion of a photographing device.

7. The magnifying glass according to claim 1, wherein the light radiation unit comprises:
a doughnut-shaped light emission board including a plurality of first light emission parts formed on an outer layer so as to be spaced apart from each other by a predetermined distance, the plurality of first light emission parts being configured to simultaneously emit light in response to a first light emission signal from the light emission controller and a plurality of second light emission parts formed on an inner layer formed inside the outer layer so as to be spaced apart from each other by a predetermined distance, the plurality of second light emission parts being configured to simultaneously emit light in response to a second light emission signal from the light emission controller;
a second polarizer located in a direction in which light emitted by the first light emission parts located on the outer layer is radiated or in a direction in which light emitted by the second light emission parts located on the inner layer is radiated, the second polarizer constituting a polarization axis set in a second direction perpendicular to the first direction defined by the first polarizer; and
a light radiation unit housing in which the light emission board and the second polarizer are mounted.

8. The magnifying glass according to claim 7, wherein the light radiation unit housing comprises:
a plurality of coupling projecting parts formed at a side of the light radiation unit housing so as to be spaced apart from each other by a predetermined distance such that an end of the optical unit housing is detachably coupled to the plurality of coupling projecting parts;
a light emission hole formation part having a plurality of light emission holes formed inside the light radiation unit housing at positions corresponding to the plurality of first light emission parts and the plurality of second light emission parts of the light emission board, the plurality of light emission holes being formed so as to be spaced apart from each other by a predetermined distance; and
a plurality of seating projecting parts formed at the observer side of the light emission hole formation part so as to be spaced apart from each other by a predetermined distance, the plurality of seating projecting parts being configured to seat the second polarizer.

9. The magnifying glass according to claim 7, wherein cross-polarization is provided by the first polarizer and the second polarizer in order to remove diffuse reflection occurring on a surface of the observation target.

10. The magnifying glass according to claim 1, wherein
a button is formed at the housing, and
upon receiving a manipulation signal input through the button, the light emission controller provides a first light emission signal or a second light emission signal to the plurality of first light emission parts or to the plurality of second light emission parts in order to operate the plurality of first light emission parts or the plurality of second light emission parts.

11. The magnifying glass according to claim 1, wherein, upon receiving a manipulation signal from a smart device through wireless communication with the smart device, the light emission controller provides a first light emission signal or a second light emission signal to the plurality of first light emission parts or to the plurality of second light emission parts in response to the manipulation signal in order to operate the plurality of first light emission parts or the plurality of second light emission parts.
